(19) United States
(12) Patent Application Publication
(10) Pub. No.: US 2005/0210557 A1
(43) Pub. Date: Sep. 29, 2005

(54) H.A.L.O. hybird

(76) Inventor: Glen Michael Falconer, Auburn, WA (US)

Correspondence Address: Glen Falconer, 325 Washington Ave. So., Kent, WA, 98032, US

(21) Appl. No.: 10/811,352

(22) Filed: Mar. 25, 2004

(51) Int. Cl. A61F 9/00; A41D 13/08; B23P 15/28; B26D 3/00

(52) U.S. Cl. 2/16

(57) ABSTRACT

The instant invention known as “H.A.L.O. HYBIRD” ™ is an amusing multipurpose hand tool primarily used to manipulate fluid mass and multitask secondary related and unrelated natural and synthetic matter.

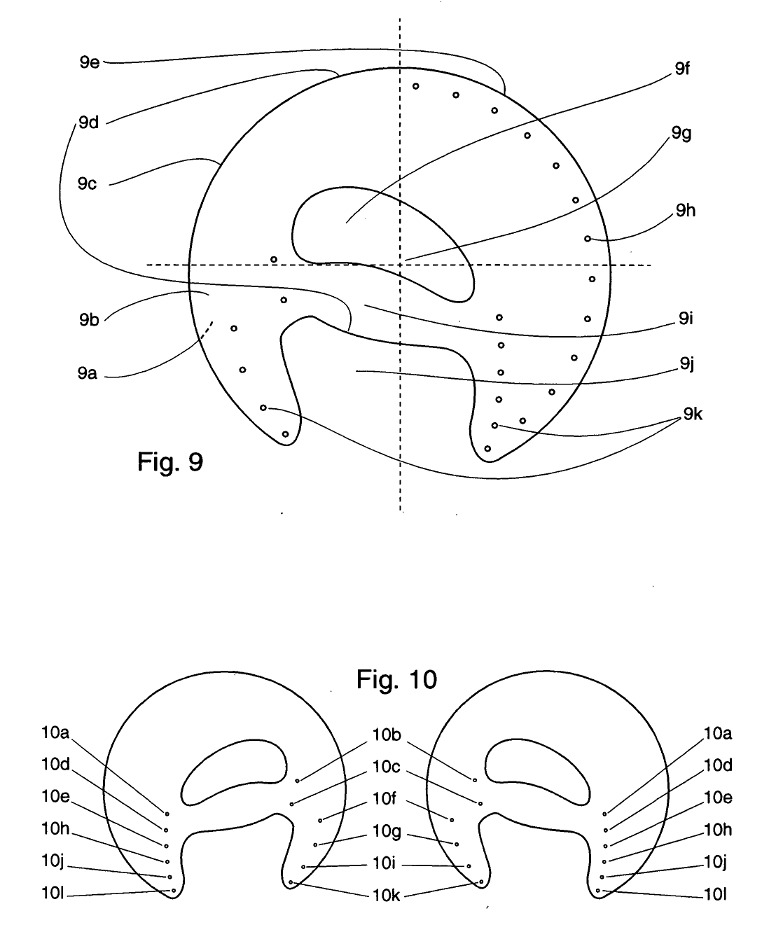

The H.A.L.O. HYBIRD™ device, (Fig. AOK) comprising a generally flat, rounded planar member which is sufficiently sized to expand the surface area of a users hand, is provided with a primary powerface (FIGS. 9a & 11a), a secondary powerface (FIGS. 9b & 11b), a leading edge (FIGS. 9c & 11c), a peripheral edge (FIG. 9d & 11d), a trailing edge (FIGS. 9e & 11e), a finger receiving aperture (FIGS. 9f & 11f), a center balance point (FIGS. 9g & 11g), a plurality of “optional” vortice producing apertures (FIGS. 9h & 11h), an integrally formed gripping region (FIGS. 9i & 11i), a hand receiving aperture bifurcating the peripheral edge (FIGS. 9j & 11j), a plurality of apertures (FIGS. 9k & 11k) facilitating a releasable hand securement means (FIGS. 5 & 17) being threadably affixed therethrough, thereby facilitating methods by which a users hand is engagably installed into said device and therein bisected, simultaneously occupying space in, on and about said planar member (FIGS. 7, 8, 19 & 20), whereby at least one finger is methodically passed through said finger receiving aperture and encouraged to work cooperatively with the thumb to multitask secondary matter (Fig. AOK).

### BACKGROUND OF THE INVENTION

The instant invention known as H.A.L.O. HYBIRD™ relates generally to hand held multipurpose utility devices which encourage multitasking secondary objects. And more generically to air engaging implements, water engaging implements, earth engaging implements, organic engaging implements, agricultural implements, martial arts implements, exercise implements, sporting goods, toys, hand tools, prosthetics and wind vanes, and provides various improvements over all known related products available in the world.

In the past, the need for human powered propulsion systems and or devices capable of manipulating fluid mass in general has caused many designers to contrive a considerable number of ill-conceived products which claim to produce desirable results . . .

To date their products provide little more than unsubstantiated, unverifiable claims eluding to: comfortable ease of use, enhanced mobility, speed, teach proper technique, reduce stresses to muscles, reduce energy expenditure, reduced pitch, yaw, wobble, oscillation, improve coordination, thought process, etc.

Such claims are paramount to asserting that a roller-skate secured to a users foot by way of a rubber band around the toes will teach proper walking technique, reduce both wobble and muscle strain while increasing blood flow to the toes, thus reducing risk of leg injury, and thereby substantially improve a users I.Q.

At the time of this writing, the inventor is unaware of any hand held device either in the prior art or currently available in the world that becomes an intuitive, integral extension of the hand, encourages multitasking secondary objects, or that produces logical and verifiable desirable results to either serious athletes or casual recreational users.

Therefore the current invention addresses the ongoing need for new and proven hand held tools capable of comfortably and efficiently multitasking fluid mass.

The inventor is aware of U.S. Pat. Nos.: U.S. Pat. No. D439,299-Chaing-01', U.S. Pat. No. 5,842,896-Liveoak98', U.S. Pat. No. 5,511,998-Johnson-96' and U.S. Pat. No. 4,913,418-Shueter-90' which generally relate to hand paddles.

With regard to U.S. Pat. No. 5,842,896-Liveoak-98':

Mr. Liveoak has developed a hand operated paddle which bares against the users hand and forearm effectively creating a “wrist splint” which eliminates a full range of motion and renders the device virtually useless to swimmers, skydivers, martial artists, kayakers, etc. Additionally, the device is constructed to be a “spoon paddle” with a positive rake and negative rake. Therefore by virtue of its design, the leading edge always digs, while the trailing edge always creates drag and turbulence. Also, the gripping region is ambidextrous and of relatively wide configuration and is not ergonomically correct in any way. Much like wearing a left shoe on the right foot, it is simply not very practical for any length of time. This of course reduces the potential of the thumb and fingers working cooperatively to perform any task other than gripping the device. Mr. Liveoak may elude to being able to grasp sticks and stones (although not in his claims) while employing his device, however, anyone (including myself) who has tried to make use of the Liveoak contraption can attest that his uncomfortable un-ergonomically correct “wrist splint” simply does not work as promised. Further exacerbating the undesirable deficiencies inherent to this design, Mr. Liveoak felt it prudent to add a neoprene type, sock like cover, which encompasses both the device and hand. This cover completely eliminates all potential for multitasking. Kayakers employing his device are unable to don helmets, adjust equipment, engage or disengage spray skirts, or liberate themselves from their trapped or submerged watercraft in an emergency situation. Fact of the matter is, that a person employing Mr. Liveoak's device will likely find him/herself presented with an opportunity to blow bubbles for the rest of their lives!

With regard to said U.S. Pat. No. D439,299-Chaing-01', U.S. Pat. No. 4,913,418-Shueter-90' and U.S. Pat. No. 5,511,998-Johnson-96':

Each of these respective designs is structured to resemble an oversized, ventilated plastic potato chip, which is secured to the palm of a users hand by way of surgical tubing wrist and finger bands. Additionally, they are all provided with wrist relief detents which bite into the wrist with every stroke.

Much like all other prior art swimming paddles, their specifications, structures, applications and claims read on each other to the extent that they all elude to: teaching proper technique, reduce injuries, reduce oscillation and wobble, enhance mobility, and of course provide a better feel of water flow.

As a retired lifeguard/swim coach, I can assure you that these “spud paddles” are about as effective as a “damp cloth” to aquatic enthusiasts. Simple tasks such as adjusting goggles and bathing suits are virtually impossible while a users hands are stuck to these uncomfortable contraptions.

The inventor is also aware of U.S. Pat. Nos.: U.S. Pat. No. 6,325,747-Norblom-01', U.S. Pat. No. 5,501,649-Queppet-96', U.S. Pat. No. D420,413-Gonzalez-00' and U.S. Pat. No. 6,036,602-Abbott-00' which generally relate to devices.

Each of these respective designs is structured to be a heavily padded sparring and training device. Each design is intended to offer users a “safe’ and “pain free”, full contact training opportunity. In contrast, my H.A.L.O. HYBIRD™ device, being provided with a sharpen-able edge, is not intended to be limited to safe or pain free sparring exercises, (if there is such a thing). Instead my H.A.L.O. HYBIRD™ device is better suited to highly skilled martial artist requiring an offensive defense capability. For example, highly skilled martial artists employing cleaving punching techniques can efficiently liberate an attackers appendages expeditiously. And because my H.A.L.O. HYBIRD™ device allows the thumbs and fingers to work cooperatively, grasping, grappling and joint locking techniques are also available options heretofore unrealized with any other hand held device.

The inventor is also aware of U.S. Pat. Nos.: U.S. Pat. No. 3,964,697-Mays-76', U.S. Pat. No. 3,969,772-Pravaz-76', U.S. Pat. No. 6,146,292-Yamanaka-00' and U.S. Pat. No. 6,368,112-Mason-02' which generally relate to skydiving devices.

The devices of Pravaz, Yamanaka, and Mason do not directly relate to hand held fluid mass manipulating implements and are intended merely to illustrate U.S. and International class and sub classification of skydiving devices,

The device of Mays however is an ill-conceived attempt to manipulate fluid mass and therefore generically illustrates my invention. The Mays device is a ludicrous contraption resembling an Icarus wing which is intended to straddle a canopy container employed by a parachutists. The notion that a patent was issued regarding this device speaks to the fact that not all patent examiners are “grumpy old men”, and clearly illustrates that some examiners have a considerable sense of humor. Even if the Mays contraption were reduced to practice, and even if the device didn't prohibit reliable deployment of a parachute, the combined surface area of the wing is so great that persons such as Arnold Schwarzeneggar would be unable to control the device to any desirable benefit. The Mays contraption is certainly obsolete, it does however serve to illustrate mankind's timeless desire to better control his/her relative space.

In contrast, skydivers employing my H.A.L.O. HYBIRD™ device are consistently clocking speeds in excess of 120 mph! Additionally, before deploying their canopies, skydivers are able to more crisply execute free flying maneuvers such as back flips, pirouettes, cartwheels, barrel rolls, headstands and corkscrews. And because the H.A.L.O. HYBIRD™ device encourages the thumbs and fingers to work cooperatively, operating parachute control systems is easily accomplished upon deployment, without the need of disengaging the hand from the device.

The inventor is also aware of U.S. Pat. Nos.: U.S. Pat. No. D255,137-Bowen-80', U.S. Pat. No. 4,324,400-Tse-82', U.S. Pat. No. 5,139,258-McPoyle-92' and U.S. Pat. No. 623,494-Mizeracki-01' which generally relate to sporting rackets.

The aforementioned devices appear to be well thought out designs, with the exception of the McPoyle device, which employs a hinged pivot between the handle and blade. Essentially, McPoyle has developed a “rubber crutch” that looks like a ping-pong paddle. Apparently, a person with poor skills is supposed to persuade a person with strong skills to use this silly contraption during competition, thereby leveling the proverbial playing field. (the term “kook” springs to mind!).

In contrast, my H.A.L.O. HYBIRD's™ device offers racketeer's some new ways to play with their balls. And because H.A.L.O. HYBIRD's™ encourage the fingers and thumb to work cooperatively, ball handlers can take paddling to the next logical level of play. H.A.L.O. HYBIRD's™ intuitively orient themselves in a natural hand shake fashion which translates into a natural, comfortable, powerful swing, forehand, backhand, overhead, sidearm and scoop.

The inventor is also aware of U.S. Pat. Nos.: U.S. Pat. No. 4,351,525-Rosenblad-82', U.S. Pat. No. 4,585,228-Olson-86', U.S. Pat. No. 5,358,463-Fuentes-94' and U.S. Pat. No. 5,530,967-Cielo-96' which generally relate to exercise weights.

Each of the aforementioned implements as well as all known prior art devices have been developed with little or no artistic flair or style. Additionally, ergonomics and creature comfort do not appear to play much of a roll with respect to exercise equipment. Most known related equipment tends to be awkward, unsightly and uncomfortable to use. The aforementioned devices do however serve to illustrate mankind's desire to train with free weights as physical fitness is in mind. In contrast, H.A.L.O. HYBIRD's™ are beautifully appointed, handsomely adorned, extremely comfortable, perfectly balanced and optionally fabricated from heavy molecular weight material which is easily provided with non-abrasive colorful coatings.

Therefore, H.A.L.O. HYBIRD's™ are well suited to T'ai-Chi, aerobics, jogging and martial arts kata because they offer a full range of well balanced motion in an often-unbalanced world.

The inventor is also aware of U.S. Pat. Nos.: U.S. Pat. No. 6,464,272-Michaud-02', U.S. Pat. No. 6,446,368-Watts-02', U.S. Pat. No. 5,678,873-Robinson-97', U.S. Pat. No. 6,113,168-St Jear-00' which generally relate to earth engaging implements.

Each of the aforementioned inventions appear to be reasonably well thought out designs which are likely to be capable of performing their intended tasks. However, as with all known prior art earth engaging implements, none are designed to become an intuitive integral extension of the human hand. Additionally, no known spade or shovel allows simultaneously performing more than one task at a time. For example: classifying clams from sand and gravel, or separating bulbs from potting soil, without the need to disengage the hand from the tool. Never before has it been possible for a person to “don” a shovel or otherwise “wear” a spade while multitasking secondary matter.

The inventor is also aware of U.S. Pat. Nos.: U.S. Pat. No. 4,378,670-Check-83', U.S. Pat. No. 5,359,840-Costar-94', U.S. Pat. No. 5,609,012-Anthes-97' and U.S. Pat. No. 5,937,627-McKittrick-99' which generally relate to gardening implements.

There appears to be no shortage of garden tools in the world. As with all known prior art, the aforementioned implements generally endeavor to create a device which resembles a “wrist splint” or “finger trap” that effectively eliminates a full range of motion. Furthermore, no known garden implement retains its orientation to a users hand while the fingers and thumb work cooperatively to multitask secondary objects. Most garden tools simply fall to the ground if a user loosens his/her grip while performing a single task.

The inventor is also aware of U.S. Pat. Nos.: U.S. Pat. No. D348,380-Allen-94', U.S. Pat. No. 3,839,788-Addis-74', U.S. Pat. No. 6,378,745-DeLuccia-02' and U.S. Pat. No. 4,087,911-Schrock-78' which generally relate to hand knives and cleavers.

A wide variety of tools have been proposed for use as knives and weapons, as well as for various industrial and agricultural implements. Each of the aforementioned inventions appear to be well suited to their intended tasks, with the exception of the DeLuccia device, which provides a hand mounted holster.

The holster being provided with a folding knife receiving pocket, which requires the use of both hands to deploy and install the knife into the “palm pocket”, which is essentially a knife-receiving sleeve. Additionally, no provision has been made to retain the knife in position within the sleeve once installed therein. In other words, a person employing this device and intending to engage in mortal combat will find it necessary to politely request his/her opponent to essentially “stand by” while I open my tiny knife, install it in my palm pocket, and form a white knuckle death grip to retain the knife in position. Additionally, if the white knuckle death grip is relaxed or released for any reason, the tiny folding knife will certainly liberate itself from the confines of its sleeve and offer its services to the opponent, who in turn will likely lodge the tiny knife in the proximity of the initial users rectal cavity where it belongs.

In contrast, persons employing my H.A.L.O. HYBIRD™ device find no necessity in gripping the device in any particular manner whatsoever. The H.A.L.O. HYBIRD™ device by virtue of its design intuitively retains itself within and about a users hand without the need of conscious thought by a user. And because the H.A.L.O. HYBIRD™ device encourages the thumb and fingers to work cooperatively, agriculturally inclined enthusiasts can for example, climb trees and in a chopping and cleaving fashion, liberate desirable food products such as bananas and coconuts without the need to sheath and unsheathe a device.

The inventor is also aware of U.S. Pat. Nos.: U.S. Pat. No. 4,080,826-Perretta-78', U.S. Pat. No. 4,522,069-Birnbaum-85', U.S. Pat. No. 4,656,770-Nuttle-87' and U.S. Pat. No. D391,875-Frontz-98' which generally relate to wind vanes.

The aforementioned, as well as all known prior art, no known wind vane has ever been adapted to govern a hand held multitasking device. The H.A.L.O. HYBIRD™ device, by virtue of its design incorporates many features and principles employed by wind vanes. The H.A.L.O. HYBIRD™ device is the only known hand held device which “trues’ itself with respect to the fluid mass presented to it. The H.A.L.O. HYBIRD™ device intuitively “trues” itself in air, water, earth, and agricultural and organic stratum. Conscious thought by a user is necessary to overcome the biased wind vane tendency of the device. Of course the strength of the wind vane tendency can be varied from a manufacturing point by adjusting the combined center of gravity, peripheral profile, surface area and vortice producing texture. The user as well can vary said tendency by selecting appropriate velocity, thrust and momentum with respect to the chosen stratum.

The inventor is also aware of U.S. Pat. Nos.: U.S. Pat. No. D386,372-Weiss-97', U.S. Pat. No. 4,324,018-Olsson-82', U.S. Pat. No. D312,379-Cassab-90' and U.S. Pat. No. 5,956,799-Panaccione-99' which generally relate to putty knives and scrapers.

As with all known prior art, none of the aforementioned implements are designed to become an intuitive integral extension of the human hand. Often tradesman are called upon to climb ladders and scaffolding or hang from a boatswain chair to perform tasks involving putty knives and scrapers. Climbing ladders and repelling boatswain chairs with H.A.L.O. HYBIRD's™ engaged is as simple as climbing and repelling without. And because H.A.L.O. HYBIRD's™ intuitively orient themselves to the hand, the likelihood of dropping one is virtually eliminated, which of course substantially reduces injuries suffered by coworkers tasking below.

The inventor is also aware of U.S. Pat. Nos.: U.S. Pat. No. D305,296-Nelson-90', U.S. Pat. No. 4,180,110-Tauscher-79', U.S. Pat. No. 4,265,285-Fodor-81' and U.S. Pat. No. 6,357,112-Bachta-02', which generally relate to hand saws.

Each of the aforementioned inventions appear to be reasonably well thought out designs which are likely to be capable of performing their intended tasks. However, as with all known prior art, none is designed to become an intuitive integral extension of the human hand. Additionally, no known handsaw retains its orientation to the hand after the hand releases its grip to the saw. While employing H.A.L.O. HYBIRD's™, both lumberjacks and tradesman can climb trees and ladders and perform their duties without the necessity of climbing like a pirate holding a knife in his/her teeth.

The inventor is also aware of U.S. Pat. Nos.: U.S. Pat. No. 3,581,740-Sherbourne-71', U.S. Pat. No. 3,942,194-Winter-76', U.S. Pat. No. 4,907,574-Hollerbach-90' and U.S. Pat. No. 5,464,444-Farquharson-95' which generally relate to medical prosthetic and rehabilitation devices.

In prosthetic devices for attachment to a forearm stump or to the hand of a person whose fingers and hands are partially or wholly paralyzed, attention generally has been paid to attempts to duplicate in one or more particulars the mechanics of a human hand. Unfortunately, little or no effort has been directed to the alleviation of the plight of aquatically inclined amputees and persons with severely restricted hand functions who wish to participate in aquatic activities such as swimming, canoeing, float tubing, aquarobics and hydrotherapy. To date no known device has been offered to handicapped persons with respect to these areas of interest. In contrast, the H.A.L.O. HYBIRD™ device encourages such persons to participate in activities such as swimming, gardening, boating, table tennis and tradesmen pursuits.

The inventor is also aware of U.S. Pat. Nos.: U.S. Pat. No. 6,131,222-Anderson-00', U.S. Pat. No. 5,829,082-Moreira-98', U.S. Pat. No. D286,501-Magan-86' and U.S. Pat. No. 4,546,510-Harrison-85' which generally relate to multipurpose hand tools.

Each of the aforementioned inventions appear to be well thought out, well designed tools capable of performing a multitude of tasks . . . The exception being the Harrison device . . . The Harrison device being a gift from a friend, who'd been unable to realize any benefit from the device, felt that because I often “tinker with things until they work” might find some use for the “second hand gift”.

After repeatedly lacerating my fingers while chopping kindling for campfires with this device, I managed to destroy a multitude of heavy leather gloves with this ill-conceived evil contraption. No matter how many times I “fixed” the sheath/handle, it always wobbled, slipped and generally flopped around uncontrollably. Eventually I wrapped the knife blade with a piece of a destroyed leather glove and encased the knife blade/sheath configuration with many rounds of electrical tape. This worked for a while, until I decided to simply pack a standard hatchet on my camping trips. Ultimately, I ended up smashing the Harrison contraption repeatedly with a 5 lb. sledge hammer before dispatching it in the general direction of my local landfill.

The inventor is also aware of U.S. Pat. Nos.: U.S. Pat. No. 23,901-Howe-94', U.S. Pat. No. 26,061-Sykes-96', U.S. Pat. No. 982,351-Cree & Davis-11' and U.S. Pat. No. 1,199,987-Husted-16' which generally relate to plates and patens.

Plates, trays and patens have been historically employed by Christian, Catholic and some Protestant clergy, to facilitate transportation and distribution of Eucharistic bread and wine during religious sacrament of communion ceremonies.

Most patens are fanciful, ornate and traditionally fabricated of heavy precious minerals such as gold, silver, brass and copper. To date no known paten has been designed or developed to resemble a simple halo that is an intuitive, integral, extension of the hand.

With respect to the other aforementioned inventions, as well as all known prior art, none are designed to be well balanced, multipurpose, intuitive, integral extensions of the human hand which is capable of manipulating elementary fluid mass and multitasking secondary matter.

With the above deficiencies in mind, I submit the following objectives of my invention for your consideration . . . .

### SUMMARY OF THE INVENTION

It is the object of the H.A.L.O. HYBIRD™ invention to provide an amusing utility device which:
1. is air engaging.
2. is water engaging.
3. is earth engaging.
4 has agricultural applications.
5. has physical fitness applications.
6. has physical therapy applications.
7. has martial arts applications.
8. has sporting goods applications.
9. offers playful amusement.
10. has clinical rehabilitation applications.
11. is easily adaptable as a medical prosthesis.
12. is a highly visible signaling device.
13. is capable of manipulating fluid mass.
14. provides powerful and predictable propulsive force.
15. is “self-truing” in a “weathervane” fashion.
16. generates vortices advantageously.
17. is stable at speeds in excess of 120 mph.
18. offers comfortable ease of use.
19. is versatile and offers multiple uses.
20. provides variant profiles to accommodate various uses.
21. is adjustable to accommodate various users.
22. utilizes available materials and is readily manufacturability.
23. becomes an intuitive integral extension of the hand.
24. encourages multitasking secondary matter.
25. is relatively light in weight and can be quickly maneuvered.
26. is constructed of sufficient structural integrity to withstand rugged use.
27. is fabricated from a desirable composite of different materials.
28. is optionally fabricated of dense, heavy molecular weight material to serve as an exercise device.
29. center of gravity can be varied during manufacture to be suitable for any number of applications.
30. provides a cutting, cleaving, scraping, sawing and chiseling blade integrally formed with an ergonomically correct handle.
31. is provided with a sharpenable edge.
32. is provided with variant saw tooth configurations.
33. is provided with variant degrees of edge temper.
34. is provided with various surface laminates, coatings and ornamentation.
35. is easily provided with a rubber band biased foam rubber ball (similar to a Child's paddleball toy).
36. provides logical and verifiable desirable results.
37. provides a novel and amusing conversation piece.

With the above and other objects in view, my invention consists in the novel construction, combination and arrangement of components heretofore and hereinafter illustrated, described, disclosed and claimed.

The instant invention known as H.A.L.O. HYBIRD™ speaks of an enhanced hand held multipurpose utility device used primarily to manipulate fluid mass and multitask secondary matter. This new and novel device presents various enhancements over all related products, either commercially available or in known prior art. This newly designed device enables users to have more than one choice of applications and offers the opportunity to perform multiple tasks simultaneously, to illustrate:

1. Skydivers employing this device are able to more crisply execute maneuvers such as back flips, pirouettes, barrel rolls, cork screws, cartwheels, etc. while utilizing this device in a “trim tab” or “air brakes” fashion, a skydiver can better play with all the sky before deploying a parachute. And because this device encourages the fingers and thumb to work cooperatively, there is no need to disengage the device in order to pull a ripcord or work parachute steering toggles, thereby multitasking while manipulating fluid mass and effectively enhancing the “fun factor”.

2. Another novel aspect of this device relates to swimmers, lifeguards for example can run, jump, dive and swim both on the surface and underwater at speeds and with power similar to that achieved with swim fins. And because this device encourages the fingers and thumbs to work cooperatively, a lifeguard can handle lines, retrieve equipment, and of course assist distressed persons, without the need to disengage the device, thereby multitasking while manipulating fluid mass and increasing the safety factor.

3. Additionally, white water play boaters employing this device find that they have “posi-traction” like performance, both on a rivers surface, as well as below a rivers surface, and can essentially “fly” their craft downstream and underwater without the bother of trying to utilize one end of a standard kayak paddle, while both ends are connected and submerged simultaneously. And because the thumbs and fingers are encouraged to work cooperatively, submerged “Squirt Boaters” for example, (occasionally) are able to pluck skipping stones from the submerged stream bed as they maneuver along, “a trophy heretofore unrealized”, thereby multitasking while manipulating fluid mass and substantially enhancing the “fun factor”.

4. Another novel aspect of this device relates to “board surfers” wanting to more efficiently accelerate their surfboards/paddleboards into position and ultimately catch more waves. Board surfers employing my H.A.L.O. HYBIRD™ device find that they are better equipped to compete with performance oriented kayaks in the surf zone. More then just a surfing safari tool, the H.A.L.O. HYBIRD™ device offers “posi-traction” like performance to “board surfers” wanting to take their sport to the next level of play. And because the hand has a full range of motion, a surfer can grab his/her surfboard, tuck the board under his/her arm, run down the beach, launch into breaking surf and paddle out past the break with heretofore-unrealized power and speed. The H.A.L.O. HYBIRD™ device is lightweight, optionally slightly buoyant, extremely durable and easily stowed to a fanny pack equipped with a simple hook and loop-fastening strap. Additionally, the H.A.L.O. HYBIRD™ device is quite handy for cracking crab legs and digging razor clams. This truly is a “wet dream” come true, which is long overdue!

5. Another novel aspect of this device relates to search and rescue mountaineering enthusiast. Such persons employing this device are able to tunnel into snow banks, either to create temporary shelter or to retrieve lost persons. And because the device may be distributed in pairs, a user is able it dig with both hands simultaneously and on any plane, or combination of planes presented in a fashion similar to the techniques employed by K-9's. And because H.A.L.O. HYBIRD's™ are provided with a wide variety of size, shape, configuration, peripheral and planar profile, edge profile and temper, they are well suited to any number of earth engaging applications. The H.A.L.O. HYBIRD™ device is lightweight, conveniently stowed and so well suited to digging that even clam digging enthusiast are able to loosen earth to the extent that clams can easily be classified from sand and gravel in order to bucket the clams for chowder stock without the need of disengaging the device, thereby multitasking while manipulating fluid mass, which of course causes no small measure of concern to the illustrious geoduck.

6. Another novel aspect of this device relates to knives and cleavers. The H.A.L.O. HYBIRD™ device being provided with a wide variety of size, shape, configuration, peripheral profile, edge profile, temper, and a sharpenable edge serves professional agricultural enthusiasts quite well. For example, harvesters and processors of cocoanuts and bananas are often called upon to climb trees and cleave loose desirable foods and bi-products. Such persons traditionally employ a machete or sickle which needs to be sheathed and unsheathed both before and after climbing. This of course is distracting and awkward. In contrast, climbing trees with H.A.L.O. HYBIRD's™ engaged is as simple as climbing without. And if a branch needs to be trimmed, all a user need do is reach out and in a “chopping fashion” liberating said branch. Additionally, sugar cane processors find utilizing this device to be quite productive, as both hands can work cooperatively, simultaneously grasping and chopping in an ambidextrous fashion, thereby multitasking fluid mass methodically.

7. Another novel aspect of this device relates to aquarobic, hydrotherapy and physical fitness enthusiast. Such persons have found that waters buoyant supportive cushion combined with its inherent omni-directional resistance provides the perfect antigravity environment for low-impact activities that are good for the whole body. Virtually everyone employing H.A.L.O. HYBIRD's™ can comfortably improve their range of motion, increase circulation, strengthen muscles, maximize flexibility and reduce “granny flaps”, by suspending weight baring joints in an aquatic environment. Aquatic exercise also allows a person to sharpen his/her “competitive edge” while reducing the likelihood of overheating and fatigue, which are usually associated with land based exercise. More than just a rehabilitation tool; the H.A.L.O. HYBIRD™ device speaks to serious and elite athletes. Ironman Olympic hopefuls for example, training with this device should expect to build upper body strength, stamina, endurance and aerobic capacity, while significantly improving mobility and their ability to multitask fluid mass.

8. Another novel aspect of this device relates to martial arts enthusiasts. Employing H.A.L.O. HYBIRD's™, a martial artist is more able to block, strike, grasp, deflect and otherwise thwart the advance of an opponent. And because the device offers a measure of shielding protection while allowing the fingers and thumbs to work cooperatively, a martial artist is more able to manipulate the fluid mass that comprises an opponent, thereby multitasking and enhancing the “success factor”.

9. Another novel aspect of this device relates to sporting rackets. Table tennis enthusiasts for example find that H.A.L.O. HYBIRD's™ offer racketeers some new ways to play with their balls. The device offers an opportunity to engage a hand paddle in a natural “hand shake” fashion heretofore unrealized. This of course translates into a natural, comfortable, powerful swing that stings the ball and sends it screaming, accurately and precisely on all planes and combination of planes presented. And because H.A.L.O. HYBIRD's™ are provided fabricated of various materials with various size, shape, configuration, weights and characteristics, they are well suited to virtually all players, skill levels and styles of play. And because the device releasably secures itself to a users hand, spectators no longer need worry about dodging high speed projectile paddles which have liberated themselves from a players grip. Additionally, H.A.L.O. HYBIRD's complement virtually every known forehand, backhand, overhead, sidearm, scoop, shave and spin technique. Also, for amusement purposes, a rubber band biased foam rubber ball may be affixed to a H.A.L.O. HYBIRD™ in such a manner as to be used in a “paddle-ball” fashion much like common children's toys where the ball returns intuitively to the paddle to be spanked again and again without contacting secondary stratum.

10. Another novel aspect of this device relates to exercise weights. Physical fitness enthusiasts for example often employ “free-weights” while exercising. Both professional athletes, Olympic hopefuls and casual “wannabees” can be found incorporating small “dumb bells” and “strap on weights” into their usual routines. To date, most such devices are awkward, uncomfortable, unbalanced and unattractive. In contrast, H.A.L.O. HYBIRD's™ are beautifully appointed, handsomely adorned, extremely comfortable, perfectly balanced and “optionally” fabricated of heavy molecular weight materials which are easily provided with non abrasive colorful coatings. And because H.A.L.O. HYBIRD's™ are provided with various sizes, shapes, configurations, colors and in various weight ranges they are well suited to pursuits such as jogging, aerobics, T'ai-Chi, Tae'-Bo and various martial arts kata. And because H.A.L.O. HYBIRD's™ offer a full range of well-balanced motion they lend themselves quite well to an often-constricted unbalanced world.

11. Another novel aspect of this device relates to gardening implements. While it's true that there appears to be no shortage of garden tools in the world, it is equally true that none are designed from the start to be perfectly balanced intuitive integral extensions of the hand. And because H.A.L.O. HYBIRD's™ are provided with various size, shape, configuration, planar profile and edge temper, gardeners are now able to employ ergonomically correct tools which encourage multitasking various matter. Edging turf, planting flowers, aerating soil, collecting leaves and twigs from planting beds and depositing same into compost bins. Classifying bulbs from potting soils and collecting tubers, mixing compost and organic type fertilizer with peat-moss and soil, digging weeds and cutting roots, raking and hoeing are all tasks which are easily and efficiently done while wearing H.A.L.O. HYBIRD's™. Additionally, every gardener knows that there is no greater marriage than hand to tool!

12. Another novel aspect of this device relates to wind vanes. To date no known hand held device has been designed from the start to be governed by wind vane features and principals. The H.A.L.O. HYBIRD™ device is the only known hand held multitasking tool which “trues” itself in air, water, earth, organic and agricultural stratum. “Conscious thought” by a user is usually necessary to overcome the biased wind vane tendency of the device. Of course the strength of the wind vane tendency can be varied from a manufacturing point by adjusting the combined center of gravity, peripheral and planar profile, surface area and vortice producing texture. The user as well can vary said tendency by selecting appropriate velocities, thrust and momentum with respect to the chosen stratum.

13. Another novel aspect of this design relates to putty knives and scrapers. Often tradesman are called upon to climb ladders and scaffolding or hang from boatswain chairs to perform tasks involving putty knives and scrapers. To date no known such implement is designed to become an intuitive integral extension of the hand. Tradesmen find that climbing ladders and repelling boatswain chairs with H.A.L.O. HYBIRD's™ engaged is as simple as climbing and repelling without. And because H.A.L.O. HYBIRD's™ intuitively orient themselves to the hand, the likelihood of dropping one during use is virtually eliminated, which of course substantially reduces injuries suffered by co-workers tasking below. And because H.A.L.O. HYBIRD's™ are provided in a variety of sizes, shapes configurations and tempers, tradesmen have wide range of choices regarding tools and applications.

14. Another novel aspect of this device relates to hand saws. While employing H.A.L.O. HYBIRD's™ lumberjacks, tradesmen, hobbyists, and back packers can climb trees, ladders, scaffolds, logjams, mountains and trails. And upon arriving at an obstacle, desired location or work station, can effectively, efficiently and safely saw, cut or trim logs, branches, roots, lumber, sheet material, pipe, conduit, bar stock, flat stock, etc.

And because H.A.L.O. HYBIRD's™ are provided in a wide variety of common sizes, shapes configurations and edge tempers, the number of applications is virtually unlimited. And because H.A.L.O. HYBIRD's™ intuitively orient themselves to both hand and stratum, a person can cut on virtually any plane or combination of planes presented. Additionally, most H.A.L.O. HYBIRD™ hand saws are provided with a KutKeeper™ configuration which resembles an oversized tooth or barb at the ends of the saw blade which helps prevent the blade from becoming dislodged during the cutting stroke.

15. Another novel aspect of this device relates to medical and rehabilitative prosthesis. In the past, a considerable number of prosthetic implements have been devised for attachment to the end of a prosthesis, most of which resemble “hooks” and “claws”. Some however, facilitate attachment of implements such as hammers, knives, screwdrivers, etc. with their focus generally being land based utilitarian pursuits. To date, little attention has been paid to aquatic activities such as swimming, canoeing, float tubing, aquarobics, etc. which are generally viewed as recreational pursuits that are of course good for the spirit and soul as well as the body.

The H.A.L.O. HYBIRD™ device being readily adaptable is easily affixed to prosthetic devices attached to the forearm stump of an amputee. This of course opens the door to a wide range of recreational and sporting pursuits heretofore unrealized. And because H.A.L.O. HYBIRD's™ are available in a wide variety if configurations such as: hand tools, sporting rackets and gardening implements, they are not limited solely to aquatic pursuits, but equally well suited to many varied hobbyists and tradesmen.

Additionally, persons who have not suffered amputation, but have limited use of their fingers and hands can benefit from H.A.L.O. HYBIRD's™ as well. For example: persons suffering from ailments such as: arthritic contracture, carpal tunnel, neuro-musculature disease, fractured bones, re-attached muscles and tendons, etc. understand that moving and flexing the muscles, joints and bones of an affected limb is a necessary part of the rehabilitation process. Without movement, the muscles, ligaments, and joints often tend to continue to contract and deteriorate, thereby curling the fingers and bending the bones. Persons incorporating H.A.L.O. HYBIRD's™ into their rehabilitation program should expect to play an active role in the rehabilitation process. H.A.L.O. HYBIRD's™ enable people to work solo and at their own pace, in the comfort of their own familiar hot tubs, pools, lakes and beaches, by gently pushing, pulling and sculling in an infinite number of directions against waters inherent omni-directional buoyant resistance. H.A.L.O. HYBIRD's™ are removably affixed intuitively in position by resilient flexible elasticized straps for frictional retention and proper orientation with respect to a wearers hand. The elastically biased straps are infinitely adjustable and bridge across the top of the fingers and hand while encompassing the wrist. Thereby gently pressing the fingers and hand into a natural “hand shake” configuration within and about the device. The fingers may be either “splay'd” out across the surface of the device or passed through the device in a gripping fashion. By “splaying” the fingers across the surface of the device, a wearer will realize the benefit of the strap gently pressing the fingers into a generally straightened position; thereby stretching affected muscles, tendons, ligaments and joints. And by pressing the device through aquatic matter perpendicularly, flexure of the wrist and hand is augmented advantageously.

Physically challenged individuals employing H.A.L.O. HYBIRD's™ in a conventional manner, find that they have substantially similar agility and effectiveness in an aquatic environment, much the same as a person without a handicap has.

In other words, H.A.L.O. HYBIRD's™ enable people with physical challenges to resume an active lifestyle by taking a pro-active role in their own rehabilitation. With practice, persistence and perseverance, a person who has a “Winners Never Quit/Quitters Never Win” attitude, may in relatively short order, find him/herself able to catch a “tasty wave”, “shred a gray cloud” and otherwise give an opponent a well deserved “good paddling”!

16. Another novel aspect of this device relates to plates and patens. Patens have historically been employed by Christian, Catholic and some Protestant clergymen to facilitate transportation and distribution of Eucharistic bread and wine during religious sacrament of communion ceremonies.

Most patens are fanciful, ornately decorated and traditionally fabricated of heavy precious minerals such as gold, silver, brass and copper. Gold and gold plated patens traditionally being used during “high-ranking” ceremonies, while silver, brass and copper are generally employed for most common day to day ceremonies.

In contrast, much like the “Holy Grail”, H.A.L.O. HYBIRD'S™ are not fabricated from precious metals or plated with same. H.A.L.O. HYBIRD'S™ are “optionally” fabricated from common, inexpensive, translucent and transparent plastic materials that resemble a “halo”, and which subliminally convey the message that “our daily bread” is at least in part, provided by a “higher power”.

Because the field of aerodynamics leads in the study and development of foil technologies, a majority of embodiments in the above disclosure are discussed in the context of airfoils, hydrofoils, and windvanes. However, the fundamental methodologies, benefits and or specific design features discussed in the above description are intended to be applicable to all foil type devices used in all fluid mass and fluid-like mediums. These mediums include air, water, earth, organic, agricultural, natural and synthetic mediums. Additionally, this invention speaks to the advancement of tools, toys, implements and devices that encourage the multitasking of secondary matter, a heretofore-unrealized exalted benefit.

### DETAILED DESCRIPTION OF CURRENT PREFERRED EMBODIMENTS

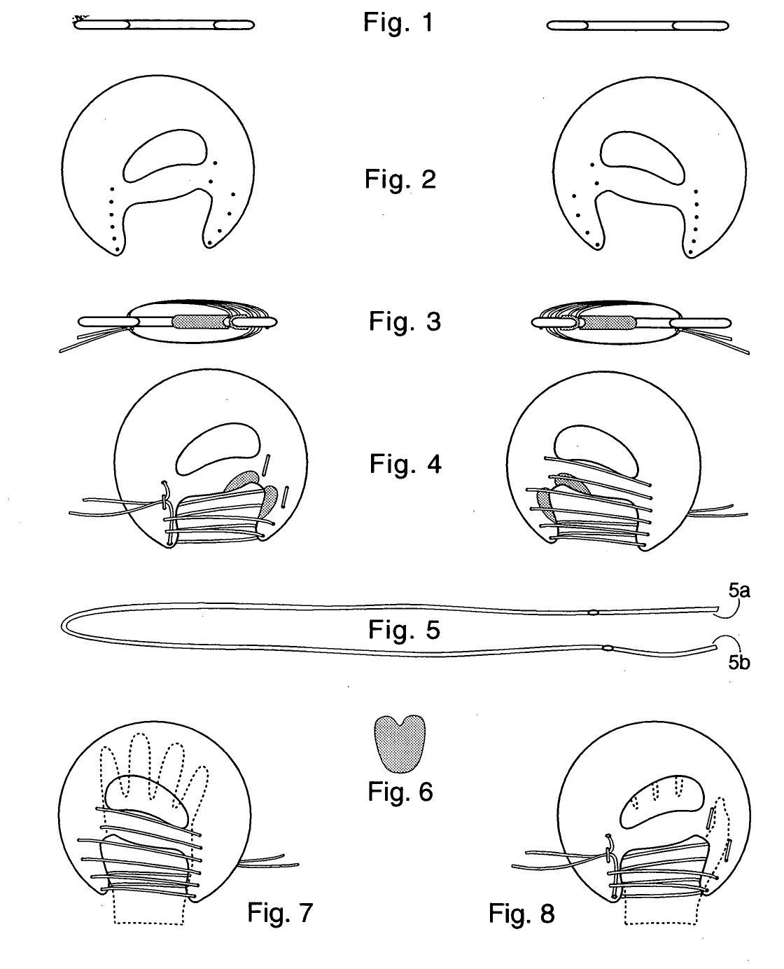

FIG. 1 is a left and right side elevational view of my H.A.L.O. HYBIRD™ device showing an unassembled condition with an aperture bifurcating the peripheral edge.

FIG. 2 is a bottom and top plan view of FIG. 1, with a plurality of irregularly shaped apertures forming an ergonomically correct gripping region and a plurality of apertures arrayed in proximity of said gripping region.

FIG. 3 is a side elevational view of FIG. 4.

FIG. 4 is a bottom and top plan view of my H.A.L.O. device in an assembled condition with adjustable, releasable cord member being threadably affixed as detailed in claim #3, and with “optional” adhesively attached heart shaped abrasion pads.

FIG. 5 is a top plan view of the cord member with a knot near each end.

FIG. 6 is a top plan view of an optional heart shaped abrasion pad.

FIG. 7 is a top plan view of FIG. 4, illustration of use, with phantom hand resting on the secondary power face.

FIG. 8 is a bottom plan view of FIG. 4, illustration of use, with phantom thumb resting on the primary power face.

FIG. 9 is an unassembled top plan view of device components comprising:
9a is the primary power face.
9b is the secondary power face.
9c is the leading edge.
9d is the peripheral edge.
9e is the trailing edge.
9f is the four finger-receiving apertures.
9g is the center balance point.
9h is a plurality of (optional) vortice producing apertures.
9i is the ergonomically correct gripping region.
9j is the hand-receiving aperture bifurcating the peripheral edge.
9k is the plurality of cord receiving apertures.

FIG. 10 is a top plan view mirror image illustration of preferred cord member routing schematic encompassing claim #3.

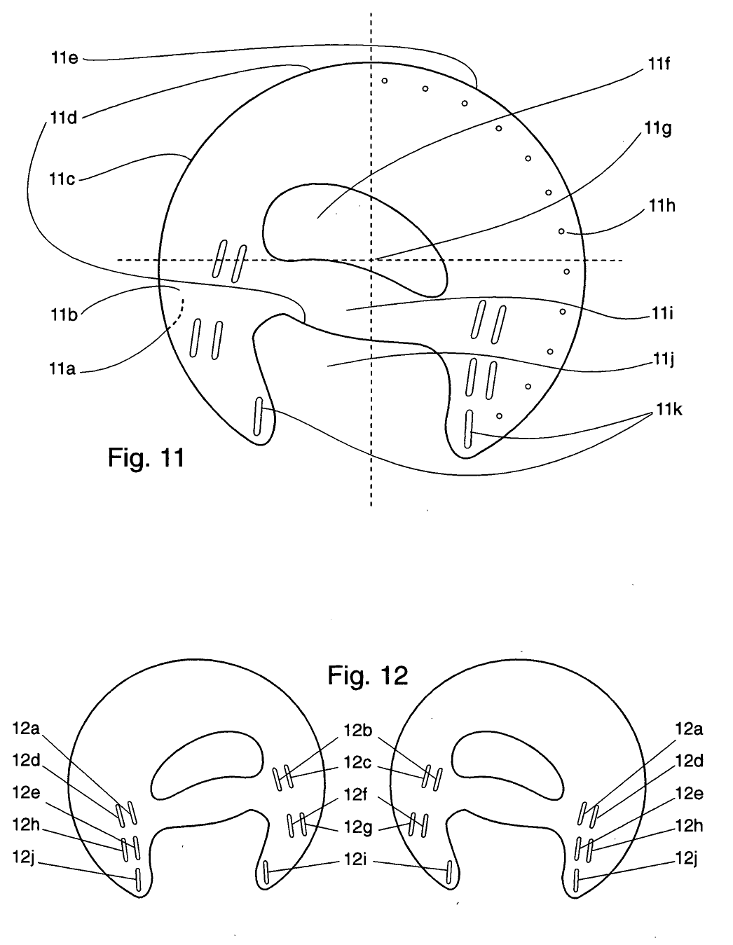

FIG. 11 is an unassembled top plan view of device components comprising:
11a is the primary powerface.
11b is the secondary powerface.
11c is the leading edge.
11d is the peripheral edge.
11e is the trailing edge.
11f is the forefinger-receiving aperture.
11g is the center balance point.
11h is a plurality of (optional) vortice producing apertures.
11i is the ergonomically correct gripping region.
11j is the hand-receiving aperture bifurcating the peripheral edge.
11k is a plurality of strap receiving apertures.

FIG. 12 is a top plan view mirror image illustration of preferred strap member routing schematic encompassing claim #5.

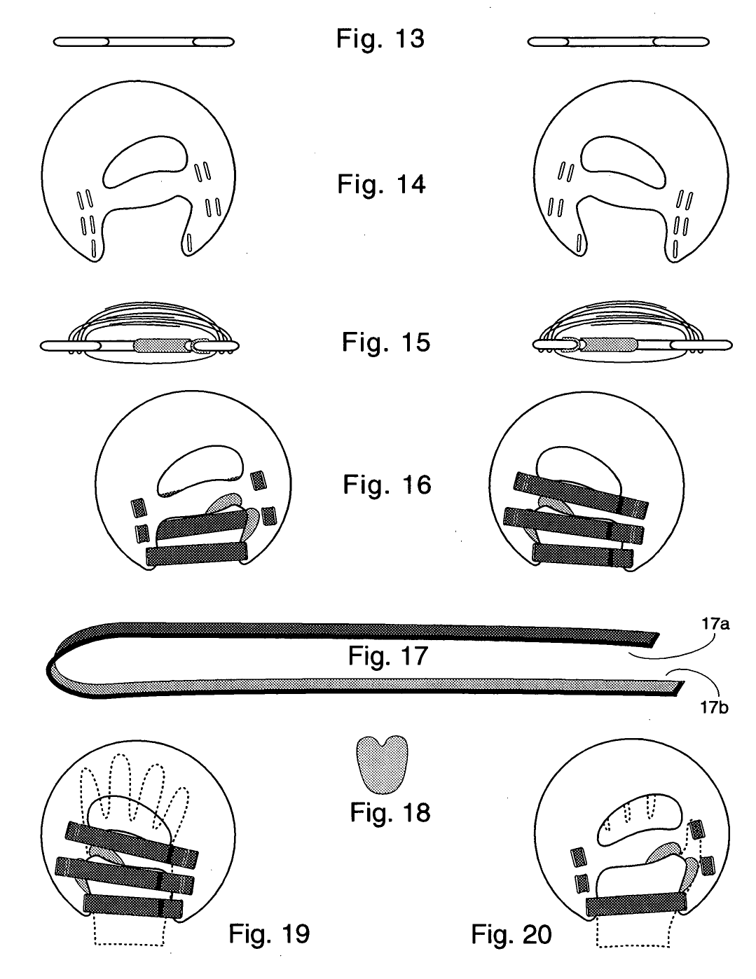

FIG. 13 is a left and right side elevational view of my H.A.L.O. HYBIRD™ device showing an unassembled condition with an aperture bifurcating the peripheral edge.

FIG. 14 is a bottom and top plan view of FIG. 1, with a plurality of irregularly shaped apertures forming an ergonomically correct gripping region and a plurality of apertures arrayed in proximity of said gripping region.

FIG. 15 is a side elevational view of FIG. 16.

FIG. 16 is a bottom and top plan view of my H.A.L.O. device in an unassembled condition with adjustable, releasable strap member being threadably affixed as detailed in claim #5, and provided with “optional” adhesively attached heart shaped abrasion pads.

FIG. 17 is a side elevational perspective view of the strap member.

FIG. 18 is a top plan view of the “optional” heart shaped abrasion pad.

FIG. 19 is a top plan view of FIG. 16, illustration of use, with phantom hand resting on secondary power face.

FIG. 20 is a bottom plan view of FIG. 16, illustration of use, with phantom thumb resting on primary powerface.

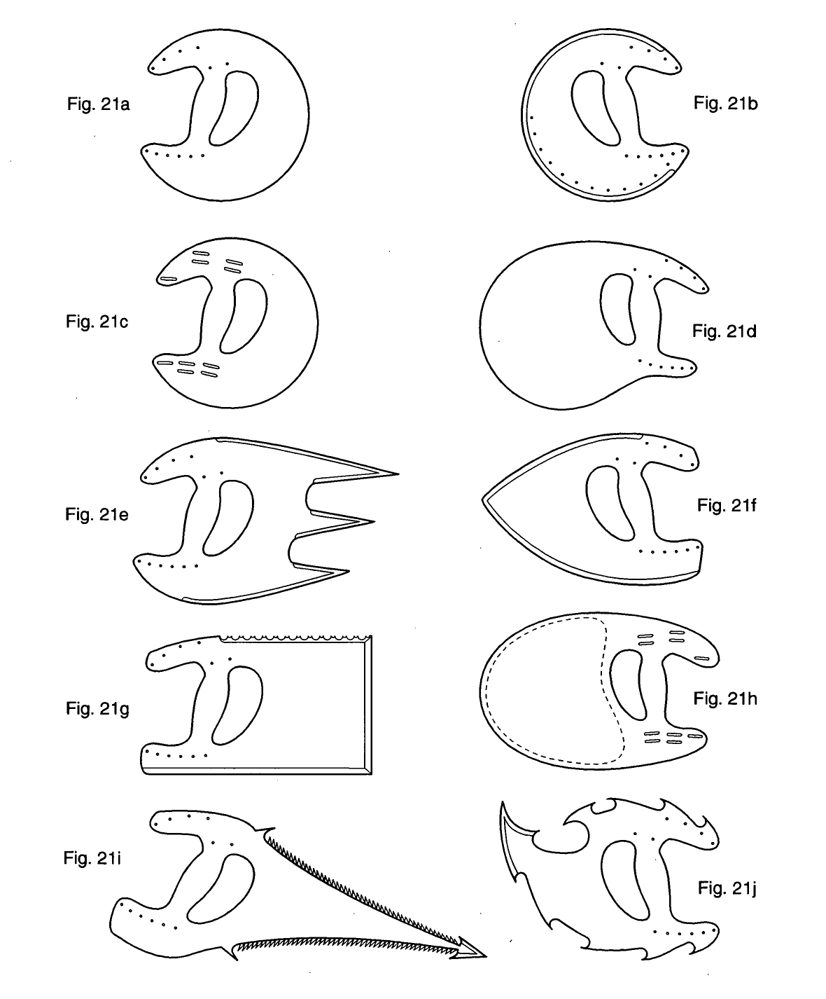

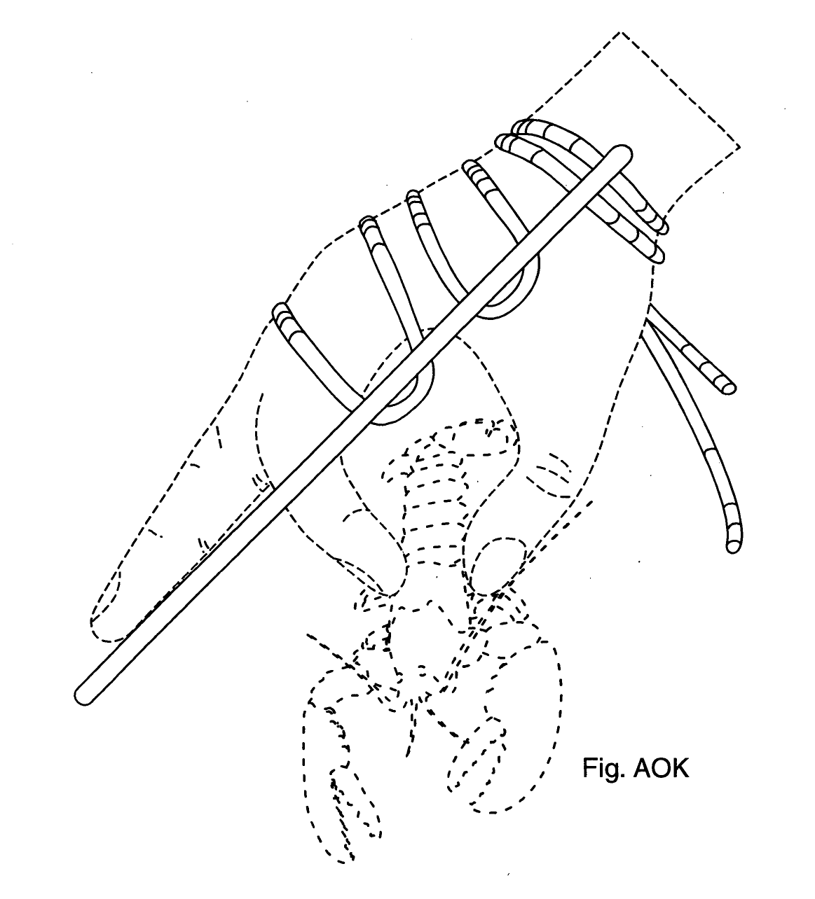

FIG. 21 is top plan views of current unassembled H.A.L.O. HYBIRD™ planar member configurations comprising:
21a HALO-HYDRO™ (rounded with radius edge).
21b HILO-HALO™ (rounded with “optional” vortice producing apertures and both a radius edge and a sharpenable edge.
21c HALO-HYDRO-WHITEWATER-MARINE™ (rounded, with hook and loop type securement system and a radius edge).
21d HALO-COASTER™ (teardrop, with a radius edge).
21e HALO-FORKIT™ (forked, with both a radius edge and a sharpenable edge).
21f HALO-EDGEMASTER™ (spade, with both a radius edge and a sharpenable edge).
21g HALO-SCRAPPER™ (squared, with a radius edge, a serrated edge and a sharpenable edge).
21h HALO-RACKETEER™ (oblong, with hook and loop securement system, a radius edge and “phantom” foam rubber like pad adhesively attached).
21i HALO-MAKO™ (triangular, hand saw with KUTKEEPER™ feature and a sharpenable edge).
21j HALO-AVALANCHE™ (flame, avalanche shovel/fantasy knife with both radius edge and a sharpenable edge).

Fig AOK is a “P.T.O.O.G.” H.A.L.O. HYBIRD™ perspective illustration side elevational view, with “phantom” hand multitasking secondary “phantom” matter.

Each of the aforementioned embodiments is designed to become a “perfectly balanced”, “self-truing”, “intuitive integral extension” of the human hand. Additionally, each design intuitively orients itself properly to a users hand and facilitates a full and complete range of motion which of course encourages the multitasking of secondary matter.

These current preferred embodiments presented may be scaled to accommodate any size hand, and may be modified to accommodate any number of applications, environments and user preferences.

Furthermore, by combining the information provided within the enclosed summary, illustrated embodiments and claims, anyone skilled in the art will be readily able to manufacture and utilize the technology herein presented by employing simple common hand tools, readily available common materials and a small measure of common sense.

And because the aforementioned embodiments are in a state of perpetual evolutionary development, no further description is possible or warranted . . .

1. An enhanced multipurpose hand tool used primarily to manipulate fluid mass and multitask secondary matter, comprising an irregularly shaped planar member encompassing a plurality of irregularly shaped apertures which provide an irregularly shaped, ergonomically correct gripping region integrally formed therebetween and within said irregularly shaped planar member which is sufficiently sized to expand the surface area of a users hand, and at least one infinitely adjustable releasable hand securement member being threadably affixed therein said planar members gripping region and encompassing at least one said aperture and a users wrist while bridging a users hand upon engagement therein, and said gripping region being offset and strategically positioned in proximity of the center balance point and the leading edge which creates a self-truing “weather vane” like effect, and at least one irregularly shaped ergonomically correct aperture bifurcating said planar members peripheral edge, and said planar member bisecting said hand upon engagement therein said ergonomically correct bifurcated peripheral edge hand receiving aperture, enabling said hand to occupy space in on and about said planar members ergonomically correct gripping region while having a full and complete range of motion, and said gripping region being provided with at least one “optional” adhesively attached abrasion pad, and said multipurpose hand tool thusly configured intuitively orienting itself through fictional engagement to both hand and matter presented, and at least one finger of said hand may be methodically inserted therethrough at least one ergonomically correct finger receiving aperture and encouraged to work cooperatively with the thumb in proximity of the primary power face to manipulate fluid mass and multitask related and unrelated secondary objects and elements such as natural and synthetic matter.

2. An enhanced multipurpose hand tool used primarily to manipulate fluid mass and multitask secondary matter, comprising an irregularly shaped planar member encomposing a plurality of irregularly shaped apertures which provide an irregularly shaped, ergonomically correct gripping region integrally formed therebetween and within said irregularly shaped planar member which is sufficiently sized to expand the surface area of a users hand, and at least one infinitely adjustable releasable hand securement member being threadably affixed therein said planar members gripping region and bridging at least one said aperture and a users hand and wrist upon engagement therein, and said gripping region being offset and strategically positioned in proximity of the center balance point and the leading edge which creates a self-truing “weather vane” like effect, and at least one irregularly shaped ergonomically correct aperture bifurcating said planar members peripheral edge, and said planar member bisecting said hand upon engagement therein said ergonomically correct bifurcated peripheral edge hand receiving aperture, enabling said hand to occupy space in on and about said planar members ergonomically correct gripping region while having a full and complete range of motion, and said gripping region being provided with at least one “optional” adhesively attached abrasion pad, and said multipurpose hand tool thusly configured intuitively orienting itself through frictional engagement to both hand and matter presented, and at least one finger of said hand may be methodically inserted therethrough at least one ergonomically correct finger receiving aperture and encouraged to work cooperatively with the thumb in proximity of the primary power face to manipulate fluid mass and multitask related and unrelated secondary objects and elements such as natural and synthetic matter.

3. A method of manipulating fluid mass and multitasking matter, comprising a pair of claim#1 planar members being laid down side by side, mirror image (FIG. 10) and a first end of cord member (FIG. 5a) is threaded down through aperture #10a and affixed with a lose knot near the end, and a second end of said cord member (FIG. 5b) is threaded down through aperture #10b, up through #10c, down through #10d, and up through #10e, down through #10f, up through #10g, down through #10h, up through #10i, down through #10j, up through #10k, down through #10l and affixed with a lose knot near the end of the cord member which hereafter encompasses the gripping region, and to assure proper fit, the hands are methodically installed therebetween said encompassing cord member and about the ergonomically correct bifurcated peripheral edge hand receiving aperture (FIG. 9j), with the finger portion of the hand resting on the secondary power face (FIG. 7), and the thumb resting on the primary power face in a “handshake” fashion (FIG. 8), which enables at least one finger to pass through said ergonomically correct finger receiving aperture (FIG. 9f), and multitask cooperatively with the thumb to grasp and pull the cord member ends simultaneously to remove excess cord from said gripping region, and upon removal of unnecessary “slack”, the thumb and fingers roll the lose knots away from said cord member ends (FIGS. 5a & 5b), and towards planar member apertures (FIGS. 10a & 10l), where each knot is “cinched down” snuggly to affix said cord member in a semi-permanent, adjustable fashion thereabout the combined hand and device configuration, which creates a right hand biased device and a left hand biased device which mirror image each other methodically, and upon assemblage, the cord member “tag ends” (FIGS. 5a & 5b) are threadably tucked under and pulled through a cord member retaining loop which has been created on the primary power face side therebetween apertures #10d & #10e, as shown in (FIG. 8) and then trimmed to suit, bearing in mind that the user may at some point in the future wish to engage a gloved hand into said device and therefore should not trim said cord member in the proximity of said knot as he/she may need to roll said knot in one direction or the other to add slack or take slack out, thus allowing the cord member to be shortened or lengthened to accommodate the size of a users hand, thus multitasking fluid mass methodically.

4. A method of manipulating fluid mass and multitasking matter comprising a pair of claim #2 planar members being laid down side by side, mirror image (FIG. 10), and a first end of said cord member (FIG. 5a) is threaded down through aperture #10a and affixed with a lose knot near the end, and a second end of said cord member (FIG. 5b) is threaded down through #10b, up through #10c, down through #10d, up through #10e, down through #10f, up through #10g, down through #10h, up through #10j, down through #10i, up through #10k, down through #10l and affixed with a lose knot near the end of said cord member which hereafter does not encompass the gripping region but rather straddles said gripping region in proximity of the secondary powerface and bridges the top of a users hand and wrist upon engagement therein without encompassing said users wrist, which allows the tag ends (FIGS. 5a & b) being threaded under and pulled through at least one cord member retaining loop created on the primary powerface side of said planar member, thus multitasking fluid mass methodically.

5. A method of manipulating fluid mass and multitasking matter, comprising a pair of planar members being laid down side by side, mirror image (FIG. 12) and a first end #17a of strap member (FIG. 17) is threaded down through aperture #12a and up through #12d and let lay temporarily, and a second end of said strap member #17b is threaded down through aperture 12b and up through #12c, enabling both said ends to be pulled equally towards the center of the gripping region and pressed together and secured together by “hook and loop” like means, and a second strap member (FIG. 17) is threadably installed by threading a first end #17a down through aperture #12e and up through #12h and let lay temporarily, and a second end of said strap member #17b is threaded down through aperture #12f and up through #12g enabling both said ends to be pulled equally towards the center of the gripping region and pressed together and secured together by “hook and loop” like means, and a third strap member (FIG. 17) is threadably installed by threading a first end #17a down through aperture #12j and up through #12i, enabling both ends to be pulled equally towards the center of the gripping region's bifurcated aperture and pressed together and secured together by “hook and loop” like means, and a users hand may be engaged therein with said strap members bridging said hand and wrist adjustably and releasably, or said strap members may bridge said hand and encompass said wrist adjustably and releasably, thus multitasking fluid mass methodically.

6. The planar member of claim #1 through #5 and #22 being fabricated of any suitable material dictated by application, environment or user preference, for example “buoyant material” or “rubber” may not necessarily be desirable in martial arts, gardening or skydiving applications.

7. The adjustable releasable hand securement means of claim #1 through #5 and #22 being fabricated of any suitable material dictated by application, environment or user preference, for example “leather” may not necessarily be desirable in hydrotherapy or rehabilitative prosthesis implements.

8. The device of claim #1 through #5 and #22 being provided with at least one “optional” adhesively attached or integrally formed abrasion pad fabricated of any suitable material dictated by application, environment or user preference, for example “felt” may not necessarily be desirable in physical fitness, aquatic or medical prosthesis applications. (FIGS. 6 & 18)

9. The device of claim #1 through #5 and #22 being provided with at least one aperture which bifurcates the peripheral edge. (FIG. 21a-j)

10. The device and components of claim #1 through #5 and #22 being provided with color, surface ornamentation, laminates or coatings as dictated by application, environment or user preference for example “adhesively attached foam rubber like sheet material” for use as a table tennis racket (FIG. 21h) or “high visibility” reflective coatings to facilitate use as a signaling device.

11. The device and components of claim #1 through #5 and #22 comprising two sides tapering to an edge, for example “knife”, chisel, serrated, saw tooth, abrasive, blunt, radius, etc. and any combination thereof as dictated by application, environment, or user preference.

12. The device and components of claim # 1 through #5 and #22 further comprising variant peripheral profiles, for example “other than round”, oblong, triangular, rectangular, forked, etc. and any combination thereof to accommodate variant applications, environments and user preferences. (FIG. 21a-j).

13. The device and components of claim #1 through #5 and #22 further comprising “optional” variant planar profiles such as “spade form”, dihedral, concave, cambered, etc. to accommodate variant applications, environments and uses, for example (FIGS. 21c & d) being provided both flat and with variant degrees of camber to facilitate stirring and mixing and transport of matter. (Not shown)

14. The planar member of claim #1 through #5 and #22 further provided with an “optional” plurality of perforations arrayed in proximity of the gripping region, to facilitate installation in any traditional fashion, “optional additional handles”, such as are common in the tang region of a knife. (Not shown)

15. The device and components of claim #1 through #5 and #22 further comprising an “optional” integrally formed, additionally contoured, more ergonomically correct gripping region such as is common with injection molding fabrication. (Not shown)

16. The device and components of claim #1 through #5 and #22 further provided with an “optional” integrally formed “lip” in proximity of the primary powerface perimeter edge, for example to facilitate retention and minimize spillage during transportation and distribution of Eucharistic bread at Christian sacrament of communion ceremonies. (Not shown).

17. The device and components of claim #1 through #5 and #22 provided with an “optional” protective sheath or padded encasement, for example to facilitate transport and/or practice of martial arts. (Not shown)

18. The device and components of claim #1 through #5 and #22 further including at least one “optional” common cord lock, clasp, strap-loop or fastener to facilitate variant hand securement means. (Not shown)

19. The device and components of claim #1 through #5 and #22 provided with variant center balance point to accommodate variant applications, environments and user preferences. (FIG. 21a-j).

20. The device and components of claim #1 through #5 and #22 provided with “optional” variant perforations, surface ornamentation and protuberances arrayed in such a manner as to add texture to the planar member, thereby creating surface tension breakers which create vortices and eliminate or substantially reduce vacuum which slows fluid mass flowing off typical drag surfaces, thereby increasing propulsion and overall performance, as dictated by application, environment or user preference, for example “texture” may not necessarily be desirable at speeds below 20 mph or in excess of mach. (FIGS. 9h & 11h).

21. The device and components of claim #1 through #5 and #22 further provided with an “optional” releasable “hook and loop” type securement means which facilitates device securement to secondary objects such as “belts”, fanny packs, safety harnesses, watercraft, tool carts, work stations, etc. (Not shown)

22. A method of manipulating fluid mass and multitasking matter comprising a hand held multipurpose device (Fig. AOK) used primarily for manipulating fluid mass and multitasking secondary matter, which comprises a generally flat, smooth, rounded, semi-ridged planar member sufficiently sized to expand the hands surface area, which is provided with at least one integrally formed, irregularly shaped, ergonomically correct, smoothly rounded finger receiving aperture (FIGS. 9f & 11f), angularly disposed slightly above the center balance point (FIGS. 9g & 11g), and offset in the general proximity of the leading edge (FIGS. 9c & 11c), and an integrally formed, irregularly shaped, ergonomically correct, smoothly rounded hand engaging aperture (FIGS. 9j & 11j), which is angularly disposed, slightly spaced apart, juxtaposed and substantially parallel in relation to said first apertures proximity (FIGS. 9 & 11), and slightly below said balance point (FIGS. 9g & 11g), and offset in the general proximity of said device leading edge (FIGS. 9c & 11c), and which bifurcates a portion of said device peripheral edge (FIGS. 9j & 11j), which flanks a portion of the users hand upon engagement therein, and an integrally formed, ergonomically correct, smoothly rounded gripping region angularly disposed therebetween said apertures (FIGS. 9i & 11i), and a hand securement means being integrally formed or affixed in such a manner as to provide a method by which a users hand is engaged and releasably secured and bisected, thus simultaneously occupying space in, on and about both primary and secondary power faces of said device in comfortable cooperation therein said gripping region (FIGS. 7, 8, 19 & 20), and said method further comprising means by which at least one of a users fingers is passed through said finger receiving aperture (FIGS. 9f & 11f), which enables it/them to work cooperatively with the thumb in proximity of the primary powerface side and perform “secondary tasks” heretofore unrealized, such as deflecting air and grasping ripcords and working parachute steering toggles (FIGS. 21a-b), and pro-pulsing water in a swimming, surfing, float tubing and boating fashion, and grasping distressed swimmers, surfboards and equipment (FIG. 21a-d), and digging snow and earth and classifying stratum and stirring, mixing and transporting composites (FIG. 21e,f & j), and climbing ladders, trees and scaffolding and cleaving, sawing and scraping matter (FIGS. 21g & i), and shielding, striking, grasping and thwarting an advancing opponent (FIG. 21a-j), and grasping, paddling, scooping and spinning balls (FIG. 21h), and pushing, pulling and lifting free weights, in an exercising fashion (FIG. 21a, b), and flexing, moving and straightening muscles, joints and bones of an affected limb in a rehabilitation process (FIG. 21a-j), and serving Eucharistic bread during Christian sacrament of communion (FIG. 21a-c), and generally manipulating related and unrelated primary and secondary objects and elements such as natural and synthetic matter (FIG. a-j), without the need to disengage said device prior to performing any secondary task (Fig. AOK), thus multitasking matter methodically.

23. The device of claim #1 through #5 being further provided with at least one “optional” rubber band biased “foam rubber” like ball being threadably affixed in such a manner that said ball intuitively returns to said device each time it is slapped, spanked and otherwise paddled by said hand/device configuration, much like a child's paddle ball toy, thus providing additional amusement to a user.
Thus different variations of my invention are provided, disclosed and claimed. The physical embodiments herein disclosed merely exemplify the invention which may be embodied in other specific structure and application, therefore, in closing, I submit that those skilled in the art will of course appreciate that my invention is in a state of perpetual evolutionary development, and that these material, shape, parameters, applications, form and detail will unquestionably evolve over time without departing from the scope and spirit of my inventive product concept and its disclosure herein. Accordingly, with inevitable evolutionary development in mind, and recognizing that additional variations of the present invention will be devised, the spirit and scope of the invention should not be limited by the embodiments illustrated and discussed, but rather by the combined complete disclosure as well as the appended claims and their legal equivalents, without departing from the inventive product concept disclosed herein. Therefore, I humbly request that my invention, disclosure and claims be interpreted as broadly as possible in hopes of precluding unethical persons from interfering with my development of my invention and derivatives thereof.
